(19) United States
(12) Patent Application Publication
(10) Pub. No.: US 2023/0026847 A1
(43) Pub. Date: Jan. 26, 2023

(54) CBD FORMULATIONS AND USES THEREOF

(71) Applicant: JUPITER WELLNESS, INC., Jupiter, FL (US)

(72) Inventor: Glynn WILSON, Jupiter, FL (US)

(21) Appl. No.: 17/937,327

(22) Filed: Sep. 30, 2022

Related U.S. Application Data
(63) Continuation of application No. 16/987,941, filed on Aug. 7, 2020, now abandoned.
(60) Provisional application No. 62/985,235, filed on Mar. 4, 2020.
(60) Provisional application No. 62/884,955, filed on Aug. 9, 2019.

(51) Int. Cl. A61K 9/06; A61P 17/08; A61K 9/00; A61K 31/05; A61K 31/167; A61K 31/352

(52) U.S. Cl. CPC A61K 9/06; A61P 17/08; A61K 9/0014; A61K 9/0053; A61K 31/05; A61K 31/167; A61K 31/352

(57) ABSTRACT

Provided herein are formulations including a cannabinoid and N-L-alpha-aspartyl-L-phenylalanine 1-methyl ester (APM). Also provided are methods of using the formulations.

### BACKGROUND

Inflammation is an important part of the immune system response to injury and infection. However, inflammation is associated with a wide variety of unwanted downstream effects, such as redness, irritation, pain, and swelling. Conditions associated with inflammation include inflammatory skin conditions such as dermatitis (e.g., atopic dermatitis), psoriasis, eczema, and poison ivy rash; joint inflammation such as from arthritis; and muscular inflammation, also referred to as myositis. Topical treatments to inhibit inflammation would be useful for such conditions, particularly when the inflammation is localized. While topically applied lidocaine may work quickly to reduce the pain associated with inflammation, it is an analgesic and thus does not act to reduce other symptoms related to inflammation such as redness or swelling. Other treatments are needed for quickly reducing inflammation-associated pain, and also reducing other symptoms of localized inflammation.

### BRIEF SUMMARY

The present disclosure provides a topical formulation comprising a cannabinoid and N-L-alpha-aspartyl-L-phenylalanine 1-methyl ester (APM) or a lower alkyl derivative thereof.

In another aspect, the present disclosure provides a method of treating a skin condition in a mammal, comprising administering to the mammal an effective amount of a composition comprising a cannabinoid and N-L-alpha-aspartyl-L-phenylalanine 1-methyl ester (APM) or a lower alkyl derivative thereof.

In another aspect, the present disclosure provides a method of alleviating skin discomfort on a mammal, comprising administering to the mammal an effective amount of a composition comprising a cannabinoid and N-L-alpha-aspartyl-L-phenylalanine 1-methyl ester (APM) or a lower alkyl derivative thereof.

In another aspect, the present disclosure provides a method of treating pain in a mammal, comprising administering to the mammal an effective amount of a composition comprising a cannabinoid and N-L-alpha-aspartyl-L-phenylalanine 1-methyl ester (APM) or a lower alkyl derivative thereof.

In another aspect, the present disclosure provides a method for treating arthritis in a mammal, comprising administering to the mammal an effective amount of a composition comprising a cannabinoid and N-L-alpha-aspartyl-L-phenylalanine 1-methyl ester (APM) or a lower alkyl derivative thereof.

In another aspect, the present disclosure provides a method of alleviating pain or discomfort in a mammal, comprising administering to the mammal an effective amount of a composition comprising a cannabinoid and N-L-alpha-aspartyl-L-phenylalanine 1-methyl ester (APM) or a lower alkyl derivative thereof.

In another aspect, the present disclosure provides a method for promoting joint health in a mammal, comprising administering to the mammal an effective amount of a composition comprising a cannabinoid and N-L-alpha-aspartyl-L-phenylalanine 1-methyl ester (APM) or a lower alkyl derivative thereof.

In another aspect, the present disclosure provides a method of reducing pain and inflammation in a mammal, comprising administering to the mammal an effective amount of a composition comprising a cannabinoid and N-L-alpha-aspartyl-L-phenylalanine 1-methyl ester (APM) or a lower alkyl derivative thereof.

Other objectives, advantages and novel features of the disclosure will become more apparent from the following detailed description.

### DETAILED DESCRIPTION

Presented herein are topical formulations comprising a cannabinoid and N-L-alpha-aspartyl-L-phenylalanine 1-methyl ester (APM) or a lower alkyl derivative thereof. Also presented are methods for treating or alleviating symptoms of skin conditions, joint conditions, and other pain and inflammation. In certain embodiments, the topical formulations are used in such methods.

In the present description, the term “about” means±20% of the indicated range, value, or structure, unless otherwise indicated. The term “consisting essentially of” limits the scope of a claim to the specified materials or steps and those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof. Any ranges provided herein include all the values and narrower ranges in the ranges.

### Cannabinoids

Provided herein are formulations and uses of formulations that include a cannabinoid and an N-L-alpha-aspartyl-L-phenylalanine 1-methyl ester (APM) or a lower alkyl derivative thereof.

The term “cannabinoid” refers to a class of compounds that bind to one or more cannabinoid receptors and act on the endocannabinoid system. Cannabinoids include phytocannabinoids, endocannabinoids, and non-naturally occurring cannabinoids. The endocannabinoid system is a biological system present in mammals that includes endocannabinoids, which are lipid based neurotransmitters that bind to cannabinoid receptors. Cannabinoid receptor 1 (CB1) and cannabinoid receptor 2 (CB2) are expressed in the central and peripheral nervous system, and cannabinoid receptor 3 (CB3) is expressed is the central nervous system. Other non-classical cannabinoid receptors include G protein-coupled receptor (GPR55), GRP119 and GPR18, peroxisome proliferator-activated receptors (PPARs) and transient receptor potential vanilloid 1 (TRPV1).

Endocannabinoid signaling through cannabinoid receptors affect cognitive processes such as mood, appetite, and memory. Cannabinoids are also present on a variety of other cells types and tissues. For example, CB2 is expressed on monocytes, macrophages, and B and T cells.

In certain embodiments, the cannabinoid is a phytocannabinoid. A phytocannabinoid is a cannabinoid that is naturally produced by a plant. Phytocannabinoids are typically C21 or C22 (for the carboxylated forms) terpenophenolic compounds. Plants that produce cannabinoids include Cannabis, Echinacea purpurea, Echinacea angustifolia, Acmelia oleracea, Helichrysum umbraculigerum, and Radula marginata. Examples of phytocannabinoids include dodeca-2E, 4E, 8Z, 10E/Z-tetraneoic-acid-isobutylamid, beta-caryophyllene, perottetinene, Δ9-Tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), cannabielsoin (CBE), and cannabicitran (CBT).

In certain embodiments, the phytocannabinoid comprises a Cannabis-derived phytocannabinoid. Cannabis generally refers to the plant genus that includes Cannabis sativa, Cannabis sativa forma indica, and Cannabis ruderalis. Examples of phytocannabinoids produced by Cannabis include Δ9-Tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), tetrahydrocannabivarinic acid (THCVA), cannabielsoin (CBE), and cannabicitran (CBT) (see, e.g., Prandi et al., Molecules 23(7), 1526, 2018). Cannabis-derived cannabinoids accumulate in secretory cavities of trichomes, which are present in the female flower of the plant. Cannabinoids may also be present in lower concentrations in seeds, roots, and stems of the plant. Many cannabis strains have either THCA or CBDA as the predominant cannabinoid produced, although it is typical for a variety of cannabinoids to be present together. When THCA and CBDA are decarboxylated, such as through heat treatment, the molecules are converted to THC and CBD, respectively.

In certain embodiments, the cannabis-derived phytocannabinoid comprises CBD. In some embodiments, the cannabis-derived phytocannabinoid comprises CBD and at least one other cannabis-derived phytocannabinoid.

In certain embodiments, the cannabinoid comprises an endocannabinoid. Endocannabinoids are lipid-based neurotransmitters that are endogenously expressed and bind to cannabinoid receptors of the endocannabinoid system. Examples of endocannabinoids include anandamide, arachidonoyl-ethanolamide (AEA), 2-arachidonoyl-glycerol (2-AG), 2-arachidonyl glyceryl ether (noladin ether), N-arachidonoyl domain (NADA), virodhamine (OAE), and lysophosphatidylinositol (LPI). In certain embodiments, the endocannabinoid comprises anandamide.

In certain specific embodiments, the cannabinoid comprises a non-naturally occurring cannabinoid (also referred to as “synthetic cannabinoid”). Examples of non-naturally occurring cannabinoids include CP55,940, which is a potent THC mimic; WIN 55,212-2 (which is an aminoalkylindole derivative with cannabinoid receptor agonist activity), nabilone (which is structurally very similar to THC), JWH-018 (1-pentyl-3-(1-naphthoyl)indole), dimethylheptylpyran, HU-210 (which is about 100 times as potent as THC), HU-331 (which is a quinone anticancinogenic drug synthesized from cannobidiol), JWH-133 (which is a potent selective CB2 receptor agonist), Levonantradol (Nantrodolum), or AM-2201 (which is a potent cannabinoid receptor agonist). In certain particular embodiments, the synthetic cannabinoid comprises CP55,940, WIN 55,212-2, or nabilone.

### N-L-alpha-aspartyl-L-phenylalanine 1-methyl ester

As previously noted, provided herein are formulations and uses of formulations that include a cannabinoid and an N-L-alpha-aspartyl-L-phenylalanine 1-methyl ester (APM) or a lower alkyl derivative thereof. N-L-alpha-aspartyl-L-phenylalanine 1-methyl ester (APM) may also be referred to as aspartame.

The term “lower alkyl derivative of APM” refers to a compound where the methyl group of the 1-methyl ester of APM is replaced with an alkyl group having 2-4 carbons, such as ethyl, propyl, isopropyl, or butyl.

### Formulations

Provided herein are formulations comprising a cannabinoid and N-L-alpha-aspartyl-L-phenylalanine 1-methyl ester (APM) or a lower alkyl derivative thereof. Such formulations include pharmaceutical formulations (also referred to as “pharmaceutical compositions”) and non-pharmaceutical formulations (also referred to “non-pharmaceutical compositions”). Proper formulation is dependent upon the route of administration chosen. Any acceptable techniques, carriers, and excipients are suitable to formulate the formulations described herein; such as those described in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999). A pharmaceutical formulation refers to a formulation for use in the treatment for a disease, disorder or condition, or for treating one or more symptoms of the disease, disorder or condition. A non-pharmaceutical formulation refers to a formulation other than a pharmaceutical formulation, such as a dietary supplement and a nutraceutical formulation.

In certain embodiments, the cannabinoid is provided in the formulation in the form of a cannabinoid isolate. The term “cannabinoid isolate” refers to a highly purified cannabis-derived cannabinoid. A cannabinoid isolate may be produced, for example, by CO2 extraction, ethanol extraction, or butane extraction. Physical forms of a cannabinoid isolate include, for example, a crystal, a powder, a wax, or a resin. A cannabinoid isolate may have a total cannabinoid content of at least 65%, at least 70%, at least 75%, at least 80%, at least 85%, at least 90%, or at least 95% cannabinoid (w/v). In certain embodiments, the cannabinoid isolate has a total cannabinoid content of at least 95% (w/v).

In some embodiments, the cannabinoid is provided in the formulation at about 0.01% to about 0.5% weight by volume (w/v). In certain embodiments, the cannabinoid is provided in the formulation at about 0.025% to about 0.5% (w/v). In certain embodiments, the cannabinoid is provided in the formulations at about 0.01% to about 0.05%, about 0.05% to about 0.1%, about 0.1% to about 0.2%, about 0.2% to about 0.3%, about 0.3% to about 0.4%, or about 0.4% to about 0.5% (w/v). Preferably, the cannabinoid is at a concentration of about 0.02% to about 0.5% (w/v), or about 0.25% to about 0.4% (w/v).

In some embodiments, the concentration of the N-L-alpha-aspartyl-L-phenylalanine 1-methyl ester (APM) or a lower alkyl derivative is about 0.05% to about 2% (w/v). In some embodiments, the concentration of the N-L-alpha-aspartyl-L-phenylalanine 1-methyl ester (APM) or a lower alkyl derivative is about 0.2 to about 2% (w/v). In some embodiments, the concentration of the N-L-alpha-aspartyl-L-phenylalanine 1-methyl ester (APM) or a lower alkyl derivative is about 0.2% to about 0.4%, about 0.4% to about 0.6%, about 0.6% to about 0.8%, about 0.8% to about 1.0%, about 1.0% to about 1.2%, about 1.2% to about 1.4%, about 1.4% to about 1.6%, about 1.6% to about 1.8%, or about 1.8% to about 2.0% (w/v). Preferably, the APM or lower alkyl derivative is at a concentration of about 0.5% to about 1.5% (w/v).

In some embodiments, the ratio of the N-L-alpha-aspartyl-L-phenylalanine 1-methyl ester or the lower alkyl derivative thereof to the cannabinoid in the formulation is in the range of about 4:1 to about 10:1 (by weight). In some embodiments, the ratio of the N-L-alpha-aspartyl-L-phenylalanine 1-methyl ester or the lower alkyl derivative thereof to the cannabinoid in the formulation is about 4:1 to about 5:1, about 5:1 to about 6:1, about 6:1 to about 7:1, about 7:1 to about 8:1, about 8:1 to about 9:1, or about 9:1 to about 10:1 (by weight). In some embodiments, the ratio of the N-L-alpha-aspartyl-L-phenylalanine 1-methyl ester or the lower alkyl derivative thereof to the cannabinoid in the formulation is in the range of about 5:1 to about 8:1 (by weight).

As used herein, “carrier” and “physiologically acceptable carriers” are used interchangeably and include any and all solvents, buffers, dispersion media, coatings, surfactants, antioxidants, preservatives (e.g., antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art and are molecular entities and compositions that are generally non-toxic to recipients at the dosages and concentrations employed, i.e., do not produce an adverse, allergic or other untoward reaction when administered to an animal, such as a human, as appropriate (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in pharmaceutical or non-pharmaceutical formulations provided herein is contemplated. In certain embodiments, the carrier is suitable to be included in topical formulations.

The formulations may comprise different types of carriers depending on whether it is to be administered in solid, liquid or aerosol form. The formulations as describe herein (and any additional active agent) can be administered intravenously, intradermally, intraarterially, intraperitoneally, intralesionally, intracranially, intraarticularly, intraprostatically, intrasplenically, intrarenally, intrapleurally, intratracheally, intranasally, intravitreally, intravaginally, intrarectally, intratumorally, intramuscularly, intraperitoneally, subcutaneously, subconjunctivally, intravesicularlly, mucosally, intrapericardially, intraumbilically, intraocularally, orally, topically, locally, by inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

In certain embodiments, the formulation is a topical formulation. A topical formulation can be in the form of, for example, solutions, suspensions, foam, lotions, gels, pastes, medicated sticks, balms (e.g., lip balm), spray, powders (e.g., body powder or baby powder), creams or ointments. Such formulations optionally contain humectants, emollients, absorption enhancing agents, solubilizers, stabilizers, tonicity enhancing agents, buffers, preservatives, and/or additional therapeutic agents.

In some embodiments, the topical formulation includes a humectant. A topical formulation can contain one or more “humectant(s)” used to provide a moistening effect. Preferably the humectant remains stable in the composition. Any suitable concentration of a single humectant or a combination of humectants can be employed, provided that the resulting concentration provides the desired moistening effect. Typically, the suitable amount of humectant will depend upon the specific humectant or humectants employed. Preferred concentration range of a single humectant or the total of a combination of humectants can be from about 0.1% to about 70%, more preferably from about 5.0% to about 30%, more specifically from about 10% to about 25% of the formulation. Non-limiting examples for use herein include glycerin, polyhydric alcohols, hyaluronic acid, and silicone oils. In certain particular embodiments, the humectant may include glycerin, propylene glycol (i.e., polypropylene glycol), glycereth-7 (a polyethylene glycol ether of glycerin), butylene glycol, sorbitol, maltitol, urea, flaxseed, algae extract, Aloe vera leaf extract, or any combination thereof.

In embodiments, the topical formulations include an emollient. A topical formulation may include an emollient to have a softening or soothing effect on the skin. In certain particular embodiments, the emollient may include glyceryl stearate, isostearyl palmitate, squalene, ceteareth-20, Simmondsia chinensis seed oil, glycol stearate, steareth-21, steareth-2, cetyl alcohol, stearyl alcohol, cetyl lactate, dimethicone, polyethylene glycol (PEG), cetearyl alcohol, ceteareth-20, PEG-100 stearate, PEG-7 glyceryl cocoate, hydroxypropyl starch phosphate, polysorbate (e.g., polysorbate 20 or polysorbate 80), dimethicone, tridecyl stearate, tridecyl trimellitate, dipentaerythrityl hexacaprylate/hexacaprate cetyl ricinoleate, C13-C14 isoparaffin, or any combination thereof. Examples of concentration ranges that the emollients may be provided in include about 0.1% to about 10%, or about 0.5% to about 5% (w/v).

In embodiments, the topical formulation may include an absorption enhancing agent. An absorption enhancing agent refers to an agent that that functions to increase absorption by enhancing membrane permeation. In certain particular embodiments, the absorption enhancing agent dimethyl isosorbide, diethylene glycol monoethyl ether, or both. Examples of concentration ranges that the absorption enhancing agents may be provided in include about 0.1% to about 10%, or about 0.5% to about 5% (w/v).

In embodiments, the topical formulation may include a preservative that exhibits antimicrobial properties. For example, preservatives can be present in a gelled formulation to minimize bacterial and/or fungal over its shelf-life. Non-limiting examples for use herein include diazolidinyl urea, methylparaben, propylparaben, butylparaben, isobutylparaben, tetrasodium EDTA, and ethylparaben. The preservative may include a combination of parabens, such as methylparaben and propylparaben. In certain specific embodiments, the preservative is merfen and thiomersal; stabilized chlorine dioxide; and quaternary ammonium compounds such as benzalkonium chloride, cetyltrimethylammonium bromide and cetylpyridinium chloride, sorbic acid, paraben, phenoxyethanol, caprylyl glycol, ethylhexylglycerin, hexylene glycol or a combination thereof. In certain embodiments, the preservative is selected from sorbic acid, paraben, phenoxyethanol, caprylyl glycol, ethylhexylglycerin, and hexylene glycol, or a combination thereof. Examples of concentration ranges that the preservatives may be provided in include about 0.1% to about 10%, or about 0.5% to about 5% (w/v).

A topical formulation of a cannabinoid and N-L-alpha-aspartyl-L-phenylalanine 1-methyl ester (APM) or a lower alkyl derivative can contain one or more “lipophilic solvent(s)”. A lipophilic solvent can be miscible with water and/or lower chain alcohols and have a vapor pressure less than water at 25° C. (˜23.8 mm Hg). A lipophilic solvent can be a glycol, specifically propylene glycol. In particular, the propylene glycol can be from the class of polyethylene glycols, specifically polyethylene glycols ranging in molecular weight from 200 to 20000. A lipophilic solvent can be from the class of glycol ethers, such as diethylene glycol monoethyl ether (transcutol, or 2-(2-ethoxyethoxy)ethanol {CAS NO 001893} or ethyoxydiglycol).

In some embodiments, the topical formulations are in the form of a suspension containing one or more polymers as suspending agents. Example polymers include water-soluble polymers such as cellulosic polymers, e.g., hydroxypropyl methylcellulose, and water-insoluble polymers such as cross-linked carboxyl-containing polymers. Certain formulations described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the topical formulations include solubilizing agents to aid in the solubility of the cannabinoids and/or the N-L-alpha-aspartyl-L-phenylalanine 1-methyl ester (APM) or a lower alkyl derivative. The term “solubilizing agent” generally includes agents that result in formation of a micellar solution or a true solution of the agent. Certain acceptable nonionic surfactants, for example polysorbate 80, are useful as solubilizing agents. Examples include glycols, polyglycols, e.g., polyethylene glycol 400, and glycol ethers.

In certain embodiments, the topical formulations include an additional active agent. The additional active agent, may for example have analgesic effects and/or anti-inflammatory effects. Examples of other additional active agents that may be included in the topical formulations include lidocaine, gorogian extract, Aloe vera leaf extra, omega fatty acids (e.g., omega-3 oil), and jojoba oil.

In certain embodiments, the additional active agent is lidocaine (or lidocaine HCL). Lidocaine is a fast-acting local anesthetic that blocks neuron signaling. A typical concentration of lidocaine is 0.1-2% (w/v). For example, a topical formulation may include about 0.5% or 0.6% lidocaine (w/v).

In certain embodiments, the additional active agent is gorgonian extract. Gorgonian extract is commercially available and is derived from Pseudopterogorgia elisabethae (commonly known as sea whip). Gorgonian extract has skin soothing and anti-inflammatory effects. In certain embodiments, the gorgonian extract is present at concentration of about 0.1% to about 1% (w/v).

In certain embodiments, the additional active agent is jojoba oil. Jojoba oil is extracted from the seeds of Simmondsia chinensis seeds, and is used for its skin soothing effects. In certain embodiments, the jojoba oil is present at concentration of about 1% to about 5% (w/v), or about 3% (w/v).

In certain embodiments, the additional active agent is Aloe vera leaf extract. Aloe is commonly used as a humectant, but also may be used for its skin soothing effects. In certain embodiments, the Aloe vera leaf extract is present at concentration of about 0.05% to about 5% (w/v), or about 0.01% (w/v).

In certain embodiments, the additional active agent is omega fatty acids (e.g., omega-3 oil), which may be present at concentration of about 0.1% to about 10% (w/v), such as about 0.1% to about 5%, or about 1% to about 10% (w/v).

A topical formulation of a cannabinoid and N-L-alpha-aspartyl-L-phenylalanine 1-methyl ester (APM) or a lower alkyl derivative can also contain a gelling agent that increases the viscosity of the final solution. The gelling agent can also act as an emulsifying agent. The formulations can form clear gels and soft gels, which upon application to the skin can break down and deteriorate, affording gels that do not dry on the skin. Typically, the concentration and combination of gelling agents will depend on the physical stability of the finished product. Preferred concentration range of a gelling agent can be from about 0.01% to about 20%, more preferably from about 0.1% to about 10%, more specifically from about 0.5% to about 5% of the formulation (w/v). Non-limiting examples for use herein include classes of celluloses, acrylate polymers and acrylate crosspolymers. Preferably, hydroxypropyl cellulose, hydroxymethyl cellulose, Pluronic PF127 polymer, carbomer 980, carbomer 1342 and carbomer 940, more preferably hydroxypropyl cellulose, Pluronic PF127 carbomer 980 and carbomer 1342, more specifically hydroxypropyl cellulose (Klucel® EF, GF and/or HF), Pluronic PF127, carbomer 980 and/or carbomer 1342 (Pemulen® TR-1, TR-2 and/or Carbopol® ETD 2020).

A topical formulation of a cannabinoid and N-L-alpha-aspartyl-L-phenylalanine 1-methyl ester (APM) or a lower alkyl derivative can contain one or more anti-oxidants, thiol containing compounds radical scavengers, and/or stabilizing agents, preferred concentration range from about 0.001% to about 0.1%, more preferably from about 0.1% to about 5% of the formulation (w/v). Non-limiting examples for use herein include butylatedhydroxytoluene, butylatedhydroxyanisole, ascorbyl palmitate, citric acid, vitamin E, vitamin E acetate, vitamin E-TPGS, ascorbic acid, sodium metabisulfite, tocophersolan and propyl gallate. More specifically the anti-oxidant can be ascorbyl palmitate, vitamin E acetate, vitamin E-TPGS, vitamin E or butylatedhydroxytoluene.

A topical formulation of a cannabinoid and N-L-alpha-aspartyl-L-phenylalanine 1-methyl ester (APM) or a lower alkyl derivative can optionally include one or more chelating agents. As used herein, the term “chelating agent” or “chelator” refers to those skin benefit agents capable of removing a metal ion from a system by forming a complex so that the metal ion cannot readily participate in or catalyze chemical reactions. The chelating agents for use herein are preferably formulated at concentrations ranging from about 0.001% to about 10%, more preferably from about 0.05% to about 5.0% of the formulation (w/v). Non-limiting examples for use herein include EDTA, disodium edeate, dipotassium edeate, cyclodextrin, trisodium edetate, tetrasodium edetate, citric acid, sodium citrate, gluconic acid and potassium gluconate. Preferably, the chelating agent can be EDTA, disodium EDTA, dipotassium EDTA, trisodium EDTA or potassium gluconate.

The topical formulation of a cannabinoid and N-L-alpha-aspartyl-L-phenylalanine 1-methyl ester (APM) or a lower alkyl derivative can be provided in any cosmetically suitable form, preferably as a gel, a lotion, or a cream, but also in an ointment or oil base, as well as a sprayable liquid form (e.g., a spray that includes the cannabinoid and N-L-alpha-aspartyl-L-phenylalanine 1-methyl ester (APM) or a lower alkyl derivative in a base, vehicle or carrier that dries in a cosmetically acceptable way without the greasy appearance that a lotion or ointment would have when applied to the skin). In certain embodiments, a topically administered formulation including a cannabinoid and N-L-alpha-aspartyl-L-phenylalanine 1-methyl ester (APM) or a lower alkyl derivative may be formulated as a balm (e.g., lip balm), a lotion, a liquid, a liquid spray (e.g., a nasal spray), or a gel. In some particular embodiments, the topically administered formulation is formulated as a gel. In other particular embodiments, the topically administered formulation is formulated as a lip balm, which may be useful, for example, for treating or alleviated symptoms associated with a cold sore.

In addition, the topical formulation can include any combination of compatible dermatologically acceptable additives commonly used, such as colorants, fragrances, and the like, as well as botanicals, such as chamomile.

In certain embodiments, a topically administered formulation including a cannabinoid and N-L-alpha-aspartyl-L-phenylalanine 1-methyl ester (APM) or a lower alkyl derivative has a pH of about 4 to about 7.5. In certain embodiments, the topical formulation has a pH of about 4 to about 4.5, about 4.5 to about 5, about 5 to about 5.5, about 5.5 to about 6, about 6 to about 6.5, or about 6.5 to about 7. Preferably, the topical formulation has a pH in the range of about 4.5 to 6.5, such as about 4.9 to about 5.1.

In some embodiments, the topical formulations include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the topical formulation in an acceptable range.

In some embodiments, the topical formulations include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, the topical formulations include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

In some embodiments, the topical formulations including the cannabinoid and N-L-alpha-aspartyl-L-phenylalanine 1-methyl ester (APM) or a lower alkyl derivative are transdermal formulations. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the cannabinoid and N-L-alpha-aspartyl-L-phenylalanine 1-methyl ester (APM) or a lower alkyl derivative is accomplished by means of iontophoretic patches and the like. In certain embodiments, transdermal patches provide controlled delivery of the cannabinoid and N-L-alpha-aspartyl-L-phenylalanine 1-methyl ester (APM) or a lower alkyl derivative. In specific embodiments, the rate of absorption is slowed by using rate-controlling membranes or by trapping the cannabinoid and N-L-alpha-aspartyl-L-phenylalanine 1-methyl ester (APM) or a lower alkyl derivative within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the cannabinoid and N-L-alpha-aspartyl-L-phenylalanine 1-methyl ester (APM) or a lower alkyl derivative optionally with carriers, optionally a rate controlling barrier to deliver the cannabinoid and N-L-alpha-aspartyl-L-phenylalanine 1-methyl ester (APM) or a lower alkyl derivative to the skin of the host at a controlled and predetermined rate over a prolonged period of time, and means to secure the device to the skin.

In topically administering the formulations including a cannabinoid and N-L-alpha-aspartyl-L-phenylalanine 1-methyl ester (APM) or a lower alkyl derivative, the skin of the mammal to be treated can be optionally pre-treated (such as washing the skin with soap and water or cleansing the skin with an alcohol-based cleanser) prior to administration of the formulation.

In certain embodiments, the topical formulation may be in form of powers, such as body powder or baby powder. The formulation may contain rice or corn micro-powder, a fragrance, and/or zinc oxide as a drying agent.

In certain embodiments, a formulation including a cannabinoid and N-L-alpha-aspartyl-L-phenylalanine 1-methyl ester (APM) or a lower alkyl derivative may be administered orally. A formulation of the invention to be orally administered can be prepared by combining a cannabinoid and N-L-alpha-aspartyl-L-phenylalanine 1-methyl ester (APM) or a lower alkyl derivative with an appropriate pharmaceutically acceptable carrier, diluent or excipient by standard methods known to one skilled in the art. The oral formulation may be in the form of liquid, tablets, powders, pills, dragees, capsules, liquids, gels, syrups, elixirs, slurries, suspensions and the like.

In some embodiments, the formulations including a cannabinoid and N-L-alpha-aspartyl-L-phenylalanine 1-methyl ester (APM) or a lower alkyl derivative are formulated for administration by inhalation. Various forms suitable for administration by inhalation include, but are not limited to, aerosols, mists or powders. Formulations of the cannabinoid and N-L-alpha-aspartyl-L-phenylalanine 1-methyl ester (APM) or a lower alkyl derivative may be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator is formulated containing a powder mix of the cannabinoid and N-L-alpha-aspartyl-L-phenylalanine 1-methyl ester (APM) or a lower alkyl derivative, and a suitable powder base such as lactose or starch. In certain embodiments, the inhalable formulation is in the form of a nasal spray.

Exemplary topical formulations may contain, in addition to CBD and AMP, one or more of the following ingredients: glyceryl stearate, isostearyl palmitate, squalene, PEG-100 stearate, cetyl ricolineate, tridecyl stearate, dipentaerythrityl hexacaprylate/hexacaprate, ceteareth-20, stearic acid, diazolidinyl urea, glycerin, dimethyl isosorbide, Aloe vera leaf extract, glycereth-7, sorbic acid, EDTA, methylparaben, propylparaben, Jojoba oil, glycol stearate, steareth-21, steareth-2, cetyl alcohol, PEG-7 glyceryl cocoate, cetyl lactate, lidocaine, dimethicone, polyacrylamide, C13-14 isoparaffin, Laureth-7, omega-3 oil, Gorgonian extract, and hyaluronic acid.

Formulations including the cannabinoid and N-L-alpha-aspartyl-L-phenylalanine 1-methyl ester (APM) or a lower alkyl derivative described herein may be manufactured by means of conventional mixing, dissolving, emulsifying, encapsulating, entrapping or lyophilizing processes. Formulations may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

Topical formulations disclosed herein may be prepared by (a) mixing hydrophilic ingredients and water to form a 1^{st} mixture, (b) mixing hydrophobic ingredients to form a 2^{nd} mixture, and (c) mixing the 1^{st} mixture and the 2^{nd} mixture together to form a 3^{rd} mixture. In step (a), AMP or a lower alkyl derivative thereof may be mixed with other hydrophilic ingredients together. Preferably, AMP is added after the other hydrophilic ingredients are already mixed together. In step (b), cannabinoid may be mixed with other hydrophobic ingredients together. Preferably, cannabinoid is added after the other hydrophobic ingredients are already mixed together. In certain other embodiments, cannabinoid may be added to the 3^{rd} mixture in a further step, step (d). Optionally, additional ingredients (e.g., a thickening agent) may be added the mixture that comprises both AMP or a lower alkyl derivative and cannabinoid in a further step, step (e).

In certain embodiments, a topical formulation is prepared by combining and mixing hydrophilic ingredients (e.g., glycerin, dimentyl isosorbide, glycereth-7, PEG-100 stearate, phenoxyethanol, methylparaben, ethylparaben, propylparaben, butylparaben, isobutylparaben, Aloe vera leaf extract (100×), hydroxypropyl starch phosphate, and/or polysorbate 20) with water. The aqueous mixture may be stirred and heated, such as at 65-80 degrees Celsius, preferably at 70-72 degrees Celsius. APM at an appropriate concentration (e.g., within the range of about 0.2% to about 2% (w/v)) may be then mixed with the aqueous mixture and stirred until dissolved. The resulting mixture (“the 1st mixture”) may similarly be heated again (if necessary).

Hydrophobic ingredients (e.g., isocetyl stearate, arlacel 165, isocetyl palmitate, Jojoba oil, tridecyl stearate, tridecyl trimellitate, dipentaerythrityl hexacaprylate/hexacaprate, PEG-7, cetearyl alcohol, ceteareth-20, cetyl ricinoleate, and/or stearic acid) may be combined and mixed. The mixture (“the 2nd mixture”) may be also stirred and heated, such as at 65-80 degrees Celsius (I., 70-72 degrees Celsius), and kept at the appropriate temperature until the mixture becomes clear and homogenous.

Next, CBD at an appropriate concentration (e.g., 0.01-0.5% (w/v)) may be mixed with the 2^{nd} mixture and then added to the 1^{st} mixture to form a composition comprising both cannabinoid and APM. Alternatively, CBD may be added after the 2^{nd} mixture is mixed with the 1^{st} mixture. The mixing of the 1^{st} mixture with the 2^{nd} mixture is preferably performed with rapid agitation, such as using a propeller stirrer, but without formation of a vortex.

The composition comprising both cannabinoid and APM should be stirred until the temperature cools to 60 degrees Celsius. At this temperature, the mixing should be switched to high shear mixing, such as with a homomixer. Next, a thickening agent (e.g., polyacrylamide (and) C13-14 isoparaffin (and) laureth-7; 1.5%) may be slowly added. High shear mixing may be continuous for an appropriate period of time, and the mixing may be switched to a gate-type mixer. The mixing may be continued until the mixture cools to a temperature of between 25 and 30 degrees Celsius. The appearance of the resulting formulation is preferably white, glossy, and viscous; the pH is preferably in the range of 4.9 and 5.1; the specific gravity is preferably in the range of 0.97 and 0.99; and the water content is preferably in the range of 50% to 61%.

### Methods of Use

Provided herein are methods of using formulations comprising a cannabinoid and N-L-alpha-aspartyl-L-phenylalanine 1-methyl ester (APM) or a lower alkyl derivative thereof as previously described.

“Mammal” includes humans and both domestic animals such as laboratory animals and household pets, (e.g. cats, dogs, swine, cattle, sheep, goats, horses, rabbits), and non-domestic animals such as wildlife and the like. In certain specific embodiments, the mammal is a human. In certain specific embodiments, the mammal is a pet, such as a dog or cat.

A “subject” according to any of the above embodiments is a mammal. Mammals include but are not limited to, domesticated animals (e.g., cows, sheep, cats, dogs, and horses), primates (e.g., human and non-human primates such as monkeys), rabbits, and rodents (e.g., mice and rats). Preferably the subject is a human. In certain embodiments, the subject does not have phenylketonuria.

“Treatment,” “treating” or “ameliorating” refers to medical management of a condition, disease, or disorder of a subject (e.g., patient) to reduce or eliminate a symptom, reduce the duration, or delay onset or progression of the condition, disease, or disorder.

An “effective amount” refers to an amount of a formulation including a cannabinoid and N-L-alpha-aspartyl-L-phenylalanine 1-methyl ester (APM) or a lower alkyl derivative thereof that provides a desired physiological change, such as an analgesic and anti-inflammatory effect. In certain embodiments, the effective amount is a therapeutically effective amount. The desired physiological change may, for example, be a decrease in symptoms of a disease, or a decrease in severity of the symptoms of the disease, or may be a reduction in the progression of symptoms of the disease. The desired physiological change may include relief from irritation, discomfort, pain, or inflammation, such as skin irritation or joint discomfort. In certain embodiments, the desired physiological change does not involve treatment of a disease.

In certain embodiments, the methods include treating a skin condition in a mammal, comprising administering to the mammal an effective amount of a composition comprising a cannabinoid and N-L-alpha-aspartyl-L-phenylalanine 1-methyl ester (APM) or a lower alkyl derivative thereof.

A skin condition as used herein refers to any disease, disorder, or injury affecting the skin. A skin condition, for example, includes chronic skin conditions, dermatological symptoms in addition to symptoms affecting other parts of the body (e.g., a Crohn's disease associated rash), lesions causes by an insect (e.g., bee stings or ant bites), a reaction to a drug (e.g., an antibiotic or an NSAID), or symptoms caused by exposure to an irritant such as poison oak or poison ivy.

In certain embodiments, the skin condition is an inflammation-associated skin condition.

In certain embodiments, the skin condition comprises eczema, atopic dermatitis, non-atopic dermatitis, psoriasis, dermatomyositis, scleroderma, seborrheic dermatitis, actinic keratosis, epidermolysis bullosa, acne, pyroderma gangrenosium, or cutaneous neoplasia.

In certain embodiments, the skin condition comprises an insect bite, poison ivy, poison oak, a chemical burn, or a radiation burn.

In particular embodiments, the skin condition comprises eczema.

In particular embodiments, the skin condition comprises dermatitis. Dermatitis refers to a broad class of skin irritation conditions. Examples of types of dermatitis include atopic dermatitis, non-atopic dermatitis, seborrheic dermatitis, follicular eczema.

In particular embodiments, the dermatitis comprises atopic dermatitis. Atopic dermatitis is also known as eczema, which is a chronic condition of red, itch flaking of the skin, often inside the elbows, behind the knees, and/or on the neck. Atopic dermatitis symptoms include erythema (i.e., reddening of the skin), induration/papulation, lichenification, and/or oozing or crusting. Lichenification refers to development of thick, dry, leathery skin patches.

In particular embodiments, the dermatitis comprises non-atopic dermatitis. Non-atopic dermatitis generally refers to dermatitis other than eczema. For example, non-atopic dermatitis includes contact allergic dermatitis.

In particular embodiments, the skin condition comprises actinic keratosis. Actinic keratosis refers to a condition characterized by rough, scaly lesions on the outer skin layer caused by chronic exposure to the ultraviolet rays of sunlight. Actinic keratosis may cause skin discomfort, and symptoms including itching and burning. Actinic keratosis lesions may become cancerous.

In certain embodiments, the skin condition comprises a cold sore, which is a small fluid-filled lesion that can occur around the mouth and is transmitted by herpes simplex virus. Cold sores may cause discomfort, pain, and/or tingling.

In certain embodiments, the methods include alleviating skin discomfort on a mammal, comprising administering to the mammal an effective amount of a composition comprising a cannabinoid and N-L-alpha-aspartyl-L-phenylalanine 1-methyl ester (APM) or a lower alkyl derivative thereof.

In certain embodiments, the skin discomfort comprises one or more symptoms associated with a rash. In certain embodiments, the skin discomfort is associated with one or more symptoms of eczema, atopic dermatitis, non-atopic dermatitis, psoriasis, dermatomyositis, scleroderma, seborrheic dermatitis, actinic keratosis, epidermolysis bullosa, acne, pyroderma gangrenosium, or cutaneous neoplasia.

In certain embodiments, the skin discomfort is associated with an insect bite, poison ivy, poison oak, a chemical burn, a thermal burn, and/or a radiation burn. The radiation burn may be a UV burn (e.g., a sunburn), an infrared burn (e.g., thermal burn), an X-ray burn, a laser-induced burn, a space travel-induced burn, or a combination thereof.

In particular embodiments, the skin discomfort is associated with a thermal burn. Thermal burns are skin injuries caused by excess heat, typically from contact with hot surfaces, hot liquids, steam or flame, or from infrared radiation without direct contact with a hot surface. The topical formulations described herein may be used, for example, for treating pain, swelling, inflammation, and/or redness associated with a thermal burn.

In certain embodiments, the symptoms associated with the rash may include symptoms comprises irritation, swelling, pain, and/or redness.

In embodiments, the methods include treating pain in a mammal, comprising administering to the mammal an effective amount of a composition comprising a cannabinoid and N-L-alpha-aspartyl-L-phenylalanine 1-methyl ester (APM) or a lower alkyl derivative thereof. In certain embodiments, the pain is skin pain, muscular pain, or joint pain.

In embodiments, the methods include treating arthritis in a mammal, comprising administering to the mammal an effective amount of a composition comprising a cannabinoid and N-L-alpha-aspartyl-L-phenylalanine 1-methyl ester (APM) or a lower alkyl derivative thereof. In certain embodiments, the arthritis is osteoarthritis, rheumatoid arthritis, or psoriatic arthritis.

In embodiments, the methods include alleviating pain or discomfort in a mammal, comprising administering to the mammal an effective amount of a composition comprising a cannabinoid and N-L-alpha-aspartyl-L-phenylalanine 1-methyl ester (APM) or a lower alkyl derivative thereof. In certain embodiments, the pain or discomfort comprises muscular pain or discomfort, or joint pain or discomfort.

In embodiments, the methods include promoting joint health in a mammal, comprising administering to the mammal an effective amount of a composition comprising a cannabinoid and N-L-alpha-aspartyl-L-phenylalanine 1-methyl ester (APM) or a lower alkyl derivative thereof. In certain embodiments, promoting joint health comprises maintaining or promoting healthy joint function.

In embodiments, the methods include reducing inflammation in a mammal, comprising administering to the mammal an effective amount of a composition comprising a cannabinoid and N-L-alpha-aspartyl-L-phenylalanine 1-methyl ester (APM) or a lower alkyl derivative thereof. In certain embodiments, the inflammation comprises skin inflammation, muscular inflammation, or joint inflammation.

In embodiments, the methods include reducing pain and inflammation in a mammal, comprising administering to the mammal an effective amount of a composition comprising a cannabinoid and N-L-alpha-aspartyl-L-phenylalanine 1-methyl ester (APM) or a lower alkyl derivative thereof. In certain embodiments, the pain and inflammation are associated with a skin condition, muscle soreness, or arthritis.

In embodiments, the methods include topically administering an effective amount of a cannabinoid and N-L-alpha-aspartyl-L-phenylalanine 1-methyl ester (APM) or a lower alkyl derivative thereof.

In embodiments, the methods include orally administering an effective amount of a cannabinoid and N-L-alpha-aspartyl-L-phenylalanine 1-methyl ester (APM) or a lower alkyl derivative thereof.

Other suitable routes of administration include, but are not limited to, intravenous, parenteral, transdermal, rectal, aerosol, ophthalmic, pulmonary, transmucosal, vaginal, otic, and nasal administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, the cannabinoid and N-L-alpha-aspartyl-L-phenylalanine 1-methyl ester (APM) or a lower alkyl derivative thereof are administered systemically. In certain embodiments, the cannabinoid and N-L-alpha-aspartyl-L-phenylalanine 1-methyl ester (APM) or a lower alkyl derivative thereof are administered in a local rather than systemic manner.

The appropriate dosage of the cannabinoid and N-L-alpha-aspartyl-L-phenylalanine 1-methyl ester (APM) or a lower alkyl derivative thereof (used alone or in combination with one or more other additional therapeutic agents) will depend on the type of disease or condition, the route of administration, body weight of the subject, severity and progression of the disease, whether the polypeptide is administered for preventive or therapeutic purposes, previous or concurrent therapeutic interventions, the subject's clinical history and response to the cannabinoid and N-L-alpha-aspartyl-L-phenylalanine 1-methyl ester (APM) or a lower alkyl derivative thereof, and the discretion of the attending physician. The practitioner responsible for administration will be able to determine the concentration of active ingredient(s) in a composition and appropriate dosing for the subject to be treated. Various dosing schedules including but not limited to single or multiple administrations over various time-points, bolus administration, and pulse infusion are contemplated herein.

The cannabinoid and N-L-alpha-aspartyl-L-phenylalanine 1-methyl ester (APM) or a lower alkyl derivative thereof may be used in an amount effective to achieve the intended purpose. For use to treat or prevent a disease condition, the cannabinoid and N-L-alpha-aspartyl-L-phenylalanine 1-methyl ester (APM) or a lower alkyl derivative thereof, or formulations thereof, are administered in a therapeutically effective amount. Determination of a therapeutically effective amount is within the capabilities of those of skill in the art, especially in light of the details provided herein.

For systemic administration, an effective amount can be estimated initially from in vitro assays, such as cell culture assays. A dose can then be formulated in animal models to achieve a circulating concentration range that includes the IC50 as determined in cell culture. Such information can be used to more accurately determine useful doses in humans. Initial dosages can also be estimated from in vivo data, e.g., animal models, using techniques that are well known in the art. Administration to humans could readily be optimized by a person of ordinary skill in the art based on animal data. Dosage amount and interval may each be adjusted to provide plasma levels the cannabinoid and N-L-alpha-aspartyl-L-phenylalanine 1-methyl ester (APM) or a lower alkyl derivative thereof which are sufficient to maintain therapeutic effect. Levels in plasma may be measured, for example, by HPLC.

In certain embodiments, the daily dosage of the formulation including the cannabinoid and N-L-alpha-aspartyl-L-phenylalanine 1-methyl ester (APM) or a lower alkyl derivative thereof ranges from about 1 μg/kg to about 100 mg/kg or more of the cannabinoid, and about 1 μg/kg to about 100 mg/kg or more of the N-L-alpha-aspartyl-L-phenylalanine 1-methyl ester (APM) or a lower alkyl derivative thereof. For repeated administrations over several days or longer, depending on the condition, the treatment may be sustained until a desired suppression of disease symptoms occurs (e.g., loss of pain, reddening, or itching). In some embodiments, a single dose of a formulation includes a range from about 0.005 mg/kg to about 10 mg/kg of the cannabinoid and about 0.001 to about 100 mg/kg of the N-L-alpha-aspartyl-L-phenylalanine 1-methyl ester (APM) or a lower alkyl derivative thereof.

In some embodiments, a topical dose may include 10-100 mg of CBD per administration.

In some embodiments, a topical dose may include 10-100 mg of N-L-alpha-aspartyl-L-phenylalanine 1-methyl ester (APM) or a lower alkyl derivative thereof per administration.

In some embodiments, an oral dose may include about 5 μg/kg/body weight to about 25 μg/kg/body weight, about 25 μg/kg/body weight to about 50 μg/kg/body weight, about 50 μg/kg/body weight to about 250 μg/kg/body weight, or about 250 μg/kg/body weight to about 500 μg/kg/body weight of cannabinoid per administration, and any range derivable therein.

In some embodiments, an oral dose may include about 5 μg/kg/body weight to about 25 μg/kg/body weight, about 25 μg/kg/body weight to about 50 μg/kg/body weight, about 50 μg/kg/body weight to about 250 μg/kg/body weight, or about 250 μg/kg/body weight to about 500 μg/kg/body weight of N-L-alpha-aspartyl-L-phenylalanine 1-methyl ester (APM) or a lower alkyl derivative thereof per administration.

Such doses may be administered intermittently, e.g., 2-3 times per day, every week, or every three weeks. An initial higher loading dose, followed by one or more lower doses may be administered. However, other dosage regimens may also be used. In certain embodiments, the formulation is a topical formulation and the formulation is topically administered at least twice a day to an affected area of skin while symptoms are occurring.

### EXAMPLES

### Example 1

### Topical Cbd Formulation for the Treatment of Atopic Dermatitis

Atopic dermatitis (AD) is one of the most common inflammatory skin diseases, affecting 13% of children and approximately 7% of adults in the United States. The disease course is chronic but intermittent, and when active, the intense pruritus and rash can be debilitating. The burden of symptoms may be profound; depression, anxiety, and sleep disturbance are frequent comorbidities (Fishbein et al., J Allergy Clin Immunol Pract. 2019 pii: S2213-2198(19)30635-X). AD is often stimulated by a cascade of inflammatory events; thus, corticosteroids immunosuppressive drugs and anti-histamines are often prescribed. However, when used chronically, these agents carry significant risk of serious adverse events. As such, there is a need for a safe and effective long-term relief of AD.

The aim of this study is to explore the efficacy of a novel cannabidiol (CBD)/aspartame lotion in the treatment of atopic dermatitis.

Three formulas are to be compared: Formula 1, which includes 500 mg/3FL OZ CBD, 1% by wt. aspartame (APM), jojoba seed oil, Aloe barbadensis leaf juice, glycerin, isocetyl stearate, glyceryl stearate, certain additional minor ingredients, and water; Formula 2, which is identical to Formula 1 except that it lacks APM (additional water is added to replace the APM); and Formula 3, which is identical to Formula 1 except that it lacks CBD and APM (additional water is added to replace the CBD and APM).

The subjects will be men and women diagnosed with chronic dermal pruritus ages 18 to 65 of any ethnicity.

The primary outcome measure is the proportion of subjects achieving success in Investigator's Static Global Assessment (ISGA) at Day 29. Success is defined as ISGA score 0 (clear) or 1 (almost clear) with at least 2 grade improvement from baseline.

The study is a double-blinded placebo controlled study. A total of 90 subjects will be enrolled. 90 subjects will be administered one of the formulas. The study will have 3 arms:

Arm 1: Subjects applying Formula 1 at least twice daily

Arm 2: Subjects applying Formula 2 at least twice daily

Arm 3: Subjects applying Formula 3 at least twice daily

Study Phase I will include enrollment and safety evaluation during the first site visit. First, potential subjects will visit the site, and the Principal Investigator (PI) will screen each potential subject for the exclusion and inclusion criteria. The PI will review each potential subject's medical history, and if the PI determines that the subject qualifies for the study, the PI will present the subject with the Informed Consent Form, and the subject will complete and sign the Informed Consent Form. Subjects will each be assigned a subject study number. The first subject will be assigned the number 001 and each subject thereafter will be assigned a consecutive number i.e., 002, 003, etc. The PI will assess and record each subject's baseline ISGA score, and will apply the assigned intervention to the subject's skin area experiencing atopic dermatitis. Each subject will be provided with a 50 mL tube (2 weeks supply) of the assigned intervention (Based on arm assignment).

Study Phase II will include daily treatment at home. For 29 days, each subject will apply as often as needed (at least twice daily) the intervention supplied by the PI.

Study Phase III will include a site visit on Day 15 (i.e., 15 days from the baseline visit). During the site visit the PI will assess and record each subject's ISGA score, and each subject will be provided with a second 50 mL tube (2 weeks supply) of the assigned intervention (Based on arm assignment).

Study Phase IV will in clue the final site visit on Day 19 (i.e., 29 days from the baseline visit). During the final site visit the PI will assess and record each subject's ISGA score.

Subjects will be randomized into 1 of 3 arms each will receive an interventional treatment: Arm 1—Formula 1; Arm 2—Formula 2; or Arm 3—Formula 3.

The duration of the study is 29 days (+/−3 days). There will be no follow-up treatment.

The following Inclusion Criteria will be used: a clinical diagnosis of AD according to the criteria of Hanifin and Rajka; has AD involvement ≥5% Treatable % BSA (excluding the scalp); has an ISGA score of Mild (2) or Moderate (3) at Baseline/Day 1; otherwise healthy subjects; male or females Ages 18-65; and able to give informed consent.

The following Exclusion Criteria will be used: as determined by the PI, a medical history that may interfere with study objectives; unstable AD or any consistent requirement for high potency topical corticosteroids; history of use of biologic therapy (including intravenous immunoglobulin); recent or anticipated concomitant use of systemic or topical therapies that might alter the course of AD; recent or current participation in another research study; females who are breastfeeding, pregnant, or with plans to get pregnant during the participation in the study; and subject is unable to provide consent or make the allotted clinical visits.

During the study, subjects will apply at home the interventional treatment i.e., arm 1, 2 or 3.

The Investigator Static Global Assessment (ISGA) score (see Table 1) is selected using the descriptors below that best describe the overall appearance of the lesions at a given time point. It is not necessary that all characteristics under Morphological Description be present.

TABLE 1
ISGA Score Descriptors

| Score | Morphological Description |
|---|---|
| 0-Clear | No inflammatory signs of atopic dermatitis |
| | (no erythema, no induration/papulation, no |
| | lichenification, no oozing/crusting). Post- |
| | inflammatory hyperpigmentation and/or |
| | hypopigmentation may be present. |
| 1-Almost | Barely perceptible erythema, barely perceptible |
| clear | induration/papulation, and/or minimal |
| | lichenification. No oozing or crusting. |
| 2-Mild | Slight but definite erythema (pink), slight |
| | but definite induration/papulation, and/or |
| | slight but definite lichenification. No oozing |
| | or crusting. |
| 3-Moderate | Clearly perceptible erythema (dull red), |
| | clearly perceptible induration/papulation, and/or |
| | clearly perceptible lichenification. Oozing and |
| | crusting may be present. |
| 4-Severe | Marked erythema (deep or bright red), marked |
| | induration/papulation, and/or marked lichenification. |
| | Disease is widespread in extent. Oozing or crusting |
| | may be present. |

During each site visit a global photograph of each subject's area of itch will be taken using a 12MP iPhone 7 (or above) camera from a distance of approximately 30 cm.

The ISGA score will be assessed by the PI. This will occur during the Phase I site visit at baseline and during the Day 15 and Day 29 site visits.

Statistical analysis will be performed to determine whether Formula 1 has a statistically significant greater efficacy than Formula 2 or Formula 3.

Primary Null Hypothesis H₀: The proportion of subjects achieving success based on ISGA score after 29 days of treatment with Formula 1 is equal to the proportion of subjects achieving success based on ISGA score after 29 days of treatment with the placebo lotion (Formula 3) or the lotion lacking aspartame (Formula 2).

Primary Alternative Hypothesis H_{A}: The proportion of subjects achieving success based on ISGA score after 29 days of treatment with Formula 1 is greater than the proportion of subjects achieving success based on ISGA score after 29 days of treatment with the Formula 3 (i.e., the placebo lotion) or Formula 2 (without aspartame).

Mathematically written for Formula 1 vs. Formula 3 as:

H₀: p_{Formula 1}−p_{Formula 3}=0

H_{A}: p_{Formula 1}−p_{Formula 3}>0

Statistical Analysis: will include Chi-squared test for proportions and Logistic regression with treatment as a factor.

The study is expected to demonstrate the greater efficacy of Formula 1, as compared to Formulas 2 and 3, in treating atopic dermatitis.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 62/884,955, filed Aug. 9, 2019 and U.S. Patent Application No. 62/985,235, filed Mar. 4, 2020, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A topical formulation, comprising a cannabinoid and N-L-alpha-aspartyl-L-phenylalanine I-methyl ester (APM) or a lower alkyl derivative thereof.

2. The topical formulation according to claim 1, wherein the cannabinoid comprises a phytocannabinoid, an endocannabinoid, or a non-naturally occurring cannabinoid.

3. The topical formulation according to claim 1, wherein the cannabinoid is a phytocannabinoid.

4. The topical formulation according to claim 3, wherein the phytocannabinoid comprises a cannabis-derived phytocannabinoid.

5. The topical formulation according to claim 4, wherein the cannabis-derived phytocannabinoid comprises one or more of Δ9-Tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), tetrahydrocannabivarinic acid (THCVA), cannabiolsoin (CBE), and cannabicitran (CBT).

6. The topical formulation according to claim 4, wherein the cannabis-derived phytocannabinoid comprises CBD.

7. The topical formulation according to claim 4, wherein the cannabis-derived phytocannabinoid comprises a cannabinoid isolate having a total cannabinoid content of at least 95% cannabinoid (w/v).

8-11. (canceled)

12. The topical formulation according to claim 1, wherein the cannabinoid is at a concentration of about 0.01% to about 0.5% weight by volume (w/v).

13. The topical formulation according to claim 1, wherein the APM or lower alkyl derivative thereof is at a concentration of about 0.05% to about 5% (w/v).

14. The topical formulation according to claim 1, wherein a ratio of the N-L-alpha-aspartyl-L-phenylalanine 1-methyl ester or the lower alkyl derivative thereof to the cannabinoid in the formulation is in the range of about 4:1 to about 10:1 (by weight).

15. The topical formulation according to claim 1, further comprising at least one of: a humectant, an emollient, an absorption enhancing agent or a preservative.

16-22. (canceled)

23. The topical formulation according to claim 1, further comprising an additional active agent.

24. The topical formulation according to claim 23 wherein the additional active agent comprises lidocaine.

25. (canceled)

26. The topical formulation according to claim 1, wherein the topical formulation is in the form of a balm, a lotion, a liquid, or a gel.

27. A method of treating a skin condition in a mammal, comprising administering to the mammal an effective amount of a composition comprising a cannabinoid and N-L-alpha-aspartyl-L-phenylalanine 1-methyl ester (APM) or a lower alkyl derivative thereof.

28. The method according to claim 27, wherein the skin condition comprises eczema, atopic dermatitis, non-atopic dermatitis, psoriasis, dermatomyositis, scleroderma, seborrheic dermatitis, actinic keratosis, epidermolysis bullosa, acne, pyroderma gangrenosium, or cutaneous neoplasia.

29. The method according to claim 27, wherein the skin condition comprises dermatitis.

30. The method according to claim 27, wherein the dermatitis comprises non-atopic dermatitis.

31-33. (canceled)

34. A method of alleviating skin discomfort on a mammal, comprising administering to the mammal an effective amount of a composition comprising a cannabinoid and N-L-alpha-aspartyl-L-phenylalanine 1-methyl ester (APM) or a lower alkyl derivative thereof.

35-52. (canceled)

53. The method according to claim 27, wherein the mammal is a human.

54. (canceled)

55. The method according to claim 27, wherein the administering comprises topically administering.

56. The method according to claim 27, wherein the administering comprises orally administering.

57-66. (canceled)
